SEQUENCE LISTING (4 sequences)

SEQ ID NO: 1 - protein, 805 aa, Homo sapiens [sequence omitted]

SEQ ID NO: 2 - protein, 672 aa, Severe acute respiratory syndrome coronavirus 2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 3 - DNA, 33 bp, Artificial
  Synthetic
aucauaggaaacaccaaagaugauauuuucuuu

SEQ ID NO: 4 - protein, 223 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

(19) United States
(12) Patent Application Publication
(10) Pub. No.: US 2023/0105667 A1
(43) Pub. Date: Apr. 6, 2023

(54) MESENCHYMAL STROMAL CELLS AND EXTRACELLULAR VESICLES FOR TREATING VIRAL INFECTIONS, INFLAMMATION, AND TISSUE FIBROSIS

(71) Applicant: EXOSTEM BIOTEC LTD., Tel Aviv (IL)

(72) Inventor: Aharon BRODIE, Meitar (IL)

(73) Assignee: EXOSTEM BIOTEC LTD., Tel Aviv (IL)

(21) Appl. No.: 17/911,056

(22) PCT Filed: Mar. 11, 2021
(86) PCT No.: PCT/IL2021/050274

§ 371 Date: Sep. 12, 2022

Related U.S. Application Data
(60) Provisional application No. 63/013,696, filed on Apr. 22, 2020.
(60) Provisional application No. 62/988,613, filed on Mar. 12, 2020.

(51) Int. Cl. A61K 35/51; A61P 31/14; A61K 31/05; A61K 31/155; C12N 15/113; A61K 35/50; A61P 13/12; A61P 11/00; A61K 45/06; A61K 9/50; C12N 9/48; C07K 14/005

(52) U.S. Cl. CPC A61K 35/51; A61P 31/14; A61K 31/05; A61K 31/155; C12N 15/1131; A61K 35/50; A61P 13/12; A61P 11/00; A61K 45/06; A61K 9/5068; C12N 9/485; C12Y 304/17023; C07K 14/005; C12N 2310/11; C12N 2310/141; C12N 2310/113; C12N 2770/20022; C12N 2770/20033

(57) ABSTRACT

The present invention provides mesenchymal stem cells (MSCs) and extracellular vesicles comprising exogenous membrane embedded proteins. Pharmaceutical compositions comprising MSCs and extracellular vesicles are also provided. The present invention further provides a method of treating, preventing or ameliorating a viral infection, inflammation, and/or tissue fibrosis.

### CROSS REFERENCE TO RELATED APPLICATIONS

This application claims the benefit of priority of U.S. Provisional Patent Application No. 62/988,613, Mar. 12, 2020 titled “MESENCHYMAL STEM CELLS AND EXTRACELLULAR VESICLES FOR TREATING VIRAL INFECTIONS”, and U.S. Provisional Patent Application No. 63/013,696 Apr. 22, 2020 titled MESENCHYMAL STROMAL CELLS AND EXTRACELLULAR VESICLES FOR TREATING VIRAL INFECTIONS, INFLAMMATION, AND TISSUE FIBROSIS, the contents of which are all incorporated herein by reference in their entirety.

### FIELD OF INVENTION

The present invention relates to mesenchymal stromal cells and extracellular vesicles and their uses for treating viral infections, inflammation, and tissue fibrosis.

### BACKGROUND OF THE INVENTION

Mesenchymal stromal cells (MSCs) are mesoderm-derived cells that can be obtained from autologous or allogeneic sources. These cells can be derived from different types of stromal cells, including bone marrow, adipose tissues and dental pulp, or from the placenta and umbilical cord. MSCs are immune privileged, expressing low levels of MHC-I and in most cases lacking expression of MHC-II.

MSCs from different sources have been widely explored for the treatment of various inflammatory and degenerative disorders and demonstrated a high safety profile. Approximately 1,000 clinical trials using human MSCs are listed in clinicaltrials.gov, out of which more than 40 have reached phase III.

MSCs have the potential to differentiate into adipogenic, chondrogenic and osteogenic lineages, in addition to some degree of myogenic differentiation upon engraftment in muscle tissue. They home to sites of inflammation and injury and have low tumorigenic potential. They were also shown to augment the activity of endogenous cells and downregulate inflammation and fibrosis.

Many of MSCs effects are mediated by a large variety of secreted factors, such as cytokines, chemokines and growth factors. In addition, recent studies demonstrated that MSCs secrete many extracellular vesicles (EVs), which can also mediate the various effects of cells by playing important roles in intercellular communication. In case of injury, MSCs attenuate tissue damage, inhibit fibrotic remodeling and apoptosis, promote angiogenesis, stimulate endogenous stem cell recruitment and proliferation and reduce immune responses.

EVs are membrane-bound nanovesicles with diameters of 30-150 nm that contain multiple proteins, nucleic acid, lipids and other molecules in a tissue- and cell-specific manner. EVs are secreted by a large variety of cells. They play major roles in cell-cell interactions and in multiple physiological and pathological conditions. EVs have been demonstrated to exert therapeutic effects in various pathological conditions via the delivery of a diverse cargo including miRNAs, lncRNAs, DNA molecules, proteins and lipids. Interestingly, similar to MSCs, EVs can home to injured, inflamed or tumor sites. In addition, EVs can internalize into various cell types and deliver both endogenous and exogenous cargos. These characteristics provide the basis for the use of EVs as drug delivery vehicles.

In addition to their therapeutic and drug delivery functions, EVs also serve as circulating biomarkers for various diseases and as mediators of disease pathogenesis. Thus, tumors or virally infected cells secrete large quantities of EVs. These are a rich source of molecules that reflect the activity of the originating cells. Due to their selective molecular packaging and stability against degrading enzymes, secreted EVs represent a viable and consistent reservoir of biomarkers in human fluids. Therefore, they emerge as attractive candidates for liquid biopsy.

MiRNAs are a family of highly conserved, small noncoding RNAs of approximately 22 nucleotides that inhibit gene expression by binding to the 3′ untranslated region (3′ UTR) of specific target mRNAs thereby inducing gene silencing. They play major roles in the regulation of various aspects of various disease development and progression. Another group of non-coding RNAs that can be used in liquid biopsy are long non-coding RNAs (lncRNAs), a class of transcripts longer than 200 nucleotides that have limited protein coding potential. lncRNAs can affect tumor growth by regulating associated gene expressions at the transcriptional, post-transcriptional or epigenetic levels and their aberrant expression has been reported as a potential measure for diagnosis and prognosis of different diseases.

SARS-CoV-2, a beta coronavirus, is the novel coronavirus that caused the COVID-19 outbreak originating in Wuhan, China in 2019. Much research is being done to develop a vaccine against the SARS-CoV-2 virus however vaccine development may be a long process and the vaccines may not be effective against future coronaviruses due to potential virus mutations.

Acute respiratory distress syndrome (ARDS) is a lung injury caused by hyper pro-inflammatory response. One of the factors that contribute to ARDS, such as in subjects infected with SARS-CoV-2, is the hyperactivation of the immune system in response to virus infection. One of the important components of the immune system that contribute to the hyperactivation of the cytokine storm are the macrophages. Lung fibrosis is considered a complication of ARDS. Similarly, other organs such as cardiac and kidney have been also reported to be affected by cytokine storm and severe hyperinflammatory responses and in chronic pathological conditions.

There is therefore a need for alternative therapeutic approaches to target microbial infection, including but not limited to, SARS-CoV-2, such as the development of therapeutics that aim to decrease viral infection, aggressiveness and patient mortality. There is also a need for alternative therapeutic approaches to treat ARDS, cytokine storm and tissue fibrosis.

### SUMMARY OF THE INVENTION

The present invention provides mesenchymal stromal cells (MSCs) and extracellular vesicles comprising exogenous proteins. Pharmaceutical compositions comprising MSCs and extracellular vesicles are also provided. The present invention further provides a method of treating, preventing or ameliorating a viral infection.

According to a first aspect, there is provided a mesenchymal stem cell (MSC) comprising an exogenous membrane associated peptide comprising a fragment of a viral peptide or virus-interacting protein.

According to another aspect, there is provided an extracellular vesicle (EV) comprising an exogenous membrane associated peptide comprising a fragment of a viral peptide or virus-interacting protein.

According to another aspect, there is provided a mesenchymal stem cell (MSC) or an extracellular vesicle (EV) derived therefrom, comprising at least one subset of molecules selected from:
(i) at least one miR or anti-miR selected from the group consisting of miR-29c, anti-miR-214 and miR-328;
(ii) at least one miR or anti-miR selected from the group consisting of miR-29c, anti-miR-214, miR-328 and miR-190;
(iii) at least one miR or anti-miR selected from the group consisting of anti-miR-181, anti-miR-214, anti-miR-1246, anti-miR-199, miR-29c, miR-27a, miR-31, miR-124, miR-127 and miR-26a;
(iv) at least one molecule selected from the group consisting of miR-29, anti-miR-214, anti-miR-21 and Eluforsen;
(v) at least one molecule selected from miR-30a, miR-9, miR-92-3p, a TRIB3 silencing molecule;
(vi) at least one miR or anti-miR selected from the group consisting of anti-miR-214, anti-miR-214, anti-miR-21, anti-miR-199, anti-miR-130, anti-miR-31, anti-miR-103, anti-miR-144, anti-miR-1825, miR-30d, miR-140p, miR-532 or miR-190;
(vii) at least one miR or anti-miR selected from the group consisting of anti-miR-214, miR-124, miR-145 or miR-27a, miR-21, anti-miR-21, miR-155, miR-20, miR-9, miR-506, miR-124, miR-455;
(viii) at least one miR or anti-miR selected from miR-532, miR-190, anti-miR-21 and anti-miR 214; and
(ix) any combination thereof.

According to some embodiments, the virus interacting protein is a virus binding receptor.

According to some embodiments, the MSC is an umbilical cord (UC) MSC or a chorionic placenta (CH) MSC.

According to some embodiments, the membrane associated peptide is a membrane embedded peptide comprising an extracellular domain of the virus interacting protein.

According to some embodiments, the fragment of a virus binding receptor is capable of binding the virus.

According to some embodiments, the virus binding receptor is a receptor employed for viral entry.

According to some embodiments, the virus is a coronavirus and the virus binding receptor is angiotensin-converting enzyme 2 (ACE2).

According to some embodiments, the ACE2 is a cleavage resistant ACE2 mutant.

According to some embodiments, ACE2 comprises the amino acid sequence of SEQ ID NO: 1 and wherein the mutant ACE2 comprises mutation of arginine 273 of SEQ ID NO: 1 to alanine.

According to some embodiments, the coronavirus is SARS-CoV-2.

According to some embodiments, the exogenous membrane associated protein comprises an extracellular fragment of a viral protein priming protein.

According to some embodiments, the viral protein is a virus spike protein.

According to some embodiments, the fragment of a viral protein priming protein is capable of priming the viral protein.

According to some embodiments, the receptor and the priming protein bind and prime the virus.

According to some embodiments, the viral protein priming protein is transmembrane protease, serine 2 (TMPRSS2).

According to some embodiments, the viral protein priming protein is an inactive mutant of the viral protein priming protein.

According to some embodiments, the viral peptide is a virus spike protein, and the fragment comprises a receptor-binding domain (RBD) of the spike protein.

According to some embodiments, the RBD comprises amino acids 437-509 of SEQ ID NO: 2.

According to some embodiments, the MSC or EV further comprises an exogenous microRNA (miR), anti-miR, small interfering RNA (siRNA), antisense oligonucleotide (ASO) or mRNA that inhibits virus replication, inhibits inflammation, inhibits fibrosis, inhibits expression of a viral protein priming protein or a combination thereof.

According to some embodiments, the viral protein priming protein is TMPRSS2 and the miR that inhibits expression of a viral protein priming protein is selected from miR-98-5p, let-7, and miR-4458.

According to some embodiments, the miR or anti-miR that inhibits inflammation is selected from: miR-124, miR-145, miR-20, miR-9, miR-506, miR-455, miR-27a, miR-29c, miR-328, miR-190, miR-532, and anti-miR-214.

According to some embodiments, the miR or anti-miR that inhibits inflammation is selected from: miR-124, miR-145, miR-29c, miR-328, miR-190, miR-532, and anti-miR-214.

According to some embodiments, the miR or anti-miR that inhibits inflammation is selected from: miR-29c, miR-328, miR-190, miR-532, and anti-miR-214.

According to some embodiments, the MSC or EV comprise exogenous miR-124 and anti-miR-214.

According to some embodiments, the MSC or EV further comprises an anti-viral agent.

According to some embodiments, the antiviral agent is selected from a vaccine, a TMPRSS2 inhibitor, an ACE2 blocking agent, soluble ACE2, and an anti-inflammatory compound.

According to some embodiments, the anti-viral agent is selected from metformin and a cannabinoid.

According to some embodiments, the cannabinoid is cannabidiol (CBD).

According to some embodiments, the MSC or EV comprises at least two different membrane associated peptides comprising a fragment of a virus interacting protein.

According to some embodiments, the extracellular vesicle is from a plant cell, a bacterial cell, an animal cell or from milk.

According to some embodiments, the animal cell is an MSC.

According to some embodiments, the MSC is an MSCs of the invention.

According to some embodiments, the MSC or EV comprises at least one miR or anti-miR selected from the group consisting of miR-29c, anti-miR-214, miR-190 and miR-328.

According to some embodiments, the MSC or EV has an anti-fibrotic effect in the lung.

According to some embodiments, the MSC or EV comprises at least one miR or anti-miR selected from the group consisting of anti-miR-214, miR-190 and miR-532.

According to some embodiments, the MSC or EV has an anti-fibrotic effect in the kidneys.

According to some embodiments, the MSC or EV comprises at least one molecule selected from the group consisting of miR-29, anti-miR-21, anti-miR-214 and/or Eluforsen.

According to some embodiments, the MSC or EV is for use in treating cystic fibrosis.

According to another aspect, there is provided a pharmaceutical composition comprising any one of:
a. an MSC of the invention,
b. an extracellular vesicle of the invention, and
c. a combination thereof.

According to some embodiments, the pharmaceutical composition further comprises a pharmaceutically acceptable carrier or adjuvant.

According to another aspect, there is provided a method of treating, preventing or ameliorating a viral infection in a subject in need thereof, the method comprising administering to the subject at least one of:
a. a pharmaceutical composition of the invention;
b. a pharmaceutical composition comprising an MSC;
c. a pharmaceutical composition comprising an extracellular vesicle from an MSC; and
d. a pharmaceutical composition comprising isolated and purified extracellular vesicles;
thereby treating, preventing or ameliorating the viral infection.

According to some embodiments, the MSC is selected from a UC MSC, and a CH MSC.

According to some embodiments, the viral infection is an infection by a virus bound by the virus binding protein.

According to some embodiments, the administering is systemic administration or administration to a site of infection.

According to some embodiments, the administration is selected from inhalation, intravenous, intranasal, intrathecal, intramuscular or oral administration.

According to some embodiments, the MSC is allogenic or autologous to the subject.

According to some embodiments, the method comprises administering a pharmaceutical composition comprising an MSC or EV of the invention.

According to some embodiments, the method further comprises administering an anti-viral medication.

According to some embodiments, the anti-viral medication is selected from a cannabinoid and metformin.

According to some embodiments, the cannabinoid is cannabidiol (CBD).

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: A bar graph of percent decrease in fluorescence in lung cells incubated with fluorescent pseudovirus particles in the presence of EVs from unmodified MSC or MSCs expressing ACE2 protein.

FIG. 2: A bar graph of relative expression of cytokines in PMA treated THP-1 cells, showing relative M1/M2 polarization. *=Pval<0.05, **=Pval<0.001, ****=Pval<0.0001.

FIG. 3: A bar graph of relative expression of cytokines in macrophages stimulated with LPS. *=Pval<0.002, **=Pval<0.001.

FIG. 4: A bar graph of the percent of cells that are fluorescent after additions of fluorescently labeled EVs.

FIG. 5: A bar graph of relative cell death of alveolar epithelial cells after treatment with LPS in the absence or presence of various MSCs or their EVs. *=Pval<0.05, **=Pval<0.01, ***=Pval<0.002, ****=Pval<0.001.

FIGS. 6A-C: Bar graphs of relative expression of (6A-B) TNFα and (6A, 6C) IL-6 in HBE cells incubated with recombinant SARS-CoV-2 peptides in the presence of (6A) MSC EVs, (6C) MSC EVs expressing miR mimics or (6B) in transwell culture with MSCs. *=Pval<0.05, **=Pval<0.01, ***=Pval<0. 001.

FIG. 7: Bar graph of relative TNFα expression in microglial cells treated with recombinant SARS-CoV-2 S1 peptide in the presence of EVs from MSCs. *=Pval<0.01.

FIGS. 8A-C: Bar graphs of relative TNFα expression in (8A) HBE cells and (8B-C) microglial cells treated with recombinant SARS-CoV-2 S1 peptide in the presence of CH-MSCs (8B) with or without the combination of an additional anti-viral agents or (8C) with the antiviral agent loaded into the EVs.

FIGS. 9A-B: Bar graphs of relative TGFβ expression in (9A) kidney fibroblasts treated with recombinant SARS-CoV-2 S1 peptide in the presence or absence of unmodified EVs or EVs loaded with miR-532, miR-190 or an anti-miR-214 as well as unmodified CH-MSCs or CH-MSCs expressing miR-190 and (9B) lung fibroblasts treated with recombinant SARS-CoV-2 S1 peptide in the presence or absence of EVs loaded with miR-29c, miR-328 or an anti-miR-214. *=Pval<0.05, **=Pval<0.01.

FIG. 10: Bar graph of relative luciferase expression from reporter cells treated with EVs from MSCs or milk expressing various miRs.

### DETAILED DESCRIPTION OF THE INVENTION

The present invention, in some embodiments, provides for mesenchymal stromal cells (MSCs) and extracellular vesicles (EV) comprising any one of (i) an exogenous membrane embedded protein, (ii) a specific oligonucleotide repertoire (e.g., miR, anti-miR, antisense oligonucleotide); and combination thereof. The present invention further concerns pharmaceutical compositions comprising these MSCs, extracellular vesicles or a combination thereof. Methods of treating, preventing or ameliorating viral infections are also provided.

According to some embodiments, there is provided an MSC comprising an exogenous membrane associated peptide, wherein said exogenous membrane associated peptide comprises an extracellular fragment of a viral protein or virus interacting protein.

According to some embodiments, there is provided an extracellular vesicle comprising an exogenous membrane associated peptide, wherein said exogenous membrane associated peptide comprises an extracellular fragment of a viral protein or virus interacting protein.

According to some embodiments, there is provided an MSC or an EV derived therefrom, comprising at least one subset of molecules selected from:
(i) at least one miR or anti-miR selected from the group consisting of miR-29c, anti-miR-21, anti-miR-214 and miR-328;
(ii) at least one miR or anti-miR selected from the group consisting of anti-miR-181, anti-miR-21, anti-miR-214, anti-miR-1246, anti-miR-199, miR 29c, miR-27a, miR-31, miR-124, miR-127 or miR-26a;
(iii) at least one molecule selected from the group consisting of miR-29, anti-miR-21, anti-miR-214 and Eluforsen;
(iv) at least one molecule selected from miR-30a, miR-9, miR-92-3p, a TRIB3 silencing molecule;
(v) at least one miR or anti-miR selected from the group consisting of anti-miR-21, anti-miR-214, anti-miR-199, anti-miR-130, anti-miR-31, anti-miR-103, anti-miR-144, anti-miR-1825, miR-30d, miR-140p, miR-532 or miR-190;
(vi) at least one miR or anti-miR selected from the group consisting of anti-miR-21, anti-miR-214, miR-124, miR-145 or miR-27a, miR-21, miR-155, miR-20, miR-9, miR-506, miR-124, miR-455; and
   any combination of (i)-(vi).

As used herein, the term “mesenchymal stromal cell”, “mesenchymal stem cell” or “MSC”, refers to multipotent stromal stem cells that have the ability to differentiate into osteoblasts, adipocytes, myocytes, chondroblasts, skeletal muscle cells and endothelial cells. MSC are present in the bone marrow, adipose tissue, peripheral blood, chorionic placenta (CH), amniotic placenta, umbilical cord (UC) blood, and dental pulp, among other tissues. The term “multipotent” refers to stem cells which are capable of giving rise to many cell types. In some embodiments, the MSC is derived from umbilical cord or chorionic placenta. In some embodiments, the MSC is derived from dental pulp, umbilical cord or chorionic placenta. In some embodiments, the MSC is derived from chorionic placenta. In some embodiments, the MSC is derived from umbilical cord. In some embodiments, the MSC is derived from dental pulp. In some embodiments, the MSC is derived from any one of umbilical cord and chorionic placenta. In some embodiments, the MSC is not derived from amniotic placenta. In some embodiments, MSC is derived from placenta. In some embodiments, the MSC is derived from amniotic placenta. In some embodiments, the MSC is derived from bone marrow. In some embodiments, the MSC is derived from adipose tissue. In some embodiments, the MSC is derived from umbilical cord blood. In some embodiments, the MSC is derived from peripheral blood. In some embodiments, the pharmaceutical composition is devoid of amniotic placenta MSCs. In some embodiments, the pharmaceutical composition is substantially devoid of amniotic placenta MSCs. In some embodiments, the MSC is not an adipose derived MSC. In some embodiments, the MSC is an unmodified MSC. In some embodiments, the MSC has been modified to over-express at least one exogenous molecule.

In some embodiments, the MSC is derived from a stem cell. In some embodiments, the MSC is differentiated from a stem cell. In some embodiments, the stem cell is a naturally occurring stem cell. In some embodiments, the stem cell is a human stem cell. In some embodiments, the stem cell is an adult stem cell. In some embodiments, the stem cell is an embryonic stem cell. In some embodiments, the stem cell is not an embryonic stem cell. In some embodiments, the stem cell is an umbilical cord stem cell. In some embodiments, the stem cell is a placental stem cell. In some embodiments, the stem cell is an induced pluripotent stem cell (iPSC). In some embodiments, the stem cell is a non-naturally occurring stem cell. In some embodiments, the MSC is derived from an iPSC. In some embodiments, MSC is differentiated from an iPSC.

In some embodiments, the MSC and/or its exosomes/extracellular vesicles are allogenic to the subject. In some embodiments, the MSC and/or its exosomes are autologous to the subject. In some embodiments, the MSC and/or its exosomes are allogenic or autologous to the subject. In some embodiments, the MSC and/or its exosomes do not induce an immune response in the subject. MSC and especially their exosomes and extracellular vesicles have a strong advantage as a therapeutic as they do not express MHCII molecules and do not induce an immune response. Further MSCs and their exosomes actively inhibit the immune response. CH and UC MSCs and their exosomes are particularly effective in this respect. In this way the MSCs and/or their exosomes can be used as an “off the shelf” therapeutic agent that can be administered to any subject in need thereof.

Chorionic (CH), and umbilical cord (UC) MSCs are well known in the art. In some embodiments, these MSCs or their secreted vesicles can be identified by examining the expression of various proteins, and regulatory RNA such as are described in international patent application WO/2018083700, the content of which are herein incorporated by reference in their entirety. In some embodiments, the MSCs are identified by the tissue they were isolated from.

In some embodiments, the MSC is an umbilical cord (UC) MSC. In some embodiments, the MSC is a chorionic placenta (CH) MSC.

In some embodiments, membrane associated is membrane embedded. In some embodiments, a membrane embedded peptide comprises a membranal domain. In some embodiments, a membranal domain is a transmembrane domain. In some embodiments, membrane associated is membrane anchored. In some embodiments, membrane anchored is lipid anchored. In some embodiments, the peptide is anchor via an anchoring peptide. In some embodiments, anchoring peptide is a glycosylphosphatidylinositol-linked protein (GPI). Methods of surface display of peptides within extracellular vesicles are well known in the art. Any method of surface display is envisioned by the invention. In some embodiments, the MSC expresses the exogenous peptide and expresses the exogenous peptide within its extracellular vesicles. In some embodiments, the exogenous peptide is added to an isolated and/or purified extracellular vesicle.

In some embodiments, a transmembrane domain is a membranal domain of a virus interacting protein. In some embodiments, a transmembrane domain is a transmembrane domain of a virus binding receptor. In some embodiments, the membranal domain is the native membranal domain of the protein comprising the fragment. In some embodiments, the fragment and membrane domain are from different proteins.

In some embodiments, the exogenous membrane associated peptide comprise a fragment of a virus-interacting protein. In some embodiments, the fragment is an extracellular fragment. In some embodiments, the fragment is a capable of interacting with the virus. In some embodiments, the fragment is an extracellular domain. In some embodiments, the fragment is the full extracellular domain. In some embodiments, the fragment is the extracellular domain and membranal domain. In some embodiments, the fragment is the full protein. In some embodiments, interacting is binding. In some embodiments, the fragment of a virus binding receptor is capable of binding the virus. In some embodiments, the virus binding receptor is a receptor used for viral entry. In some embodiments, the virus interacting protein is employed for viral entry. In some embodiments, the virus interacting protein is a host cell surface component recognized by the virus. In some embodiments, recognized by the virus is recognized as a gateway of entry into the cell.

In some embodiments, the virus is a coronavirus. In some embodiments, the virus binding receptor is angiotensin-converting enzyme 2 (ACE2). In some embodiments, the virus is a coronavirus, and the virus binding receptor is angiotensin-converting enzyme 2 (ACE2). In some embodiments, the virus interacting protein is transmembrane protease, serine 2 (TMPRSS2). In some embodiments, the virus interacting protein is transmembrane protease, serine 1 (TMPRSS1).

In some embodiments, the ACE2 is a mutant ACE2. In some embodiments, the mutant ACE2 is a cleavage resistant ACE2 mutant. In some embodiments, ACE2 is human ACE2. In some embodiments, human ACE2 comprises the amino acid sequence:

(SEQ ID NO: 1)
MSSSSWLLLSLVAVTAAQSTIEEQAKTFLDKFNHEA
EDLFYQSSLASWNYNTNITEENVQNMNNAGDKWSAF
LKEQSTLAQMYPLQEIQNLTVKLQLQALQQNGSSVL
SEDKSKRLNTILNTMSTIYSTGKVCNPDNPQECLLL
EPGLNEIMANSLDYNERLWAWESWRSEVGKQLRPLY
EEYVVLKNEMARANHYEDYGDYWRGDYEVNGVDGYD
YSRGQLIEDVEHTFEEIKPLYEHLHAYVRAKLMNAY
PSYISPIGCLPAHLLGDMWGRFWTNLYSLTVPFGQK
PNIDVTDAMVDQAWDAQRIFKEAEKFFVSVGLPNMT
QGFWENSMLTDPGNVQKAVCHPTAWDLGKGDFRILM
CTKVTMDDFLTAHHEMGHIQYDMAYAAQPFLLRNGA
NEGFHEAVGEIMSLSAATPKHLKSIGLLSPDFQEDN
ETEINFLLKQALTIVGTLPFTYMLEKWRWMVFKGEI
PKDQWMKKWWEMKREIVGVVEPVPHDETYCDPASLF
HVSNDYSFIRYYTRTLYQFQFQEALCQAAKHEGPLH
KCDISNSTEAGQKLFNMLRLGKSEPWTLALENVVGA
KNMNVRPLLNYFEPLFTWLKDQNKNSFVGWSTDWSP
YADQSIKVRISLKSALGDKAYEWNDNEMYLFRSSVA
YAMRQYFLKVKNQMILFGEEDVRVANLKPRISFNFF
VTAPKNVSDIIPRTEVEKAIRMSRSRINDAFRLNDN
SLEFLGIQPTLGPPNQPPVSIWLIVFGVVMGVIVVG
IVILIFTGIRDRKKKNKARSGENPYASIDISKGENN
PGFQNTDDVQTSF.

In some embodiments, the cleavage resistant ACE2 comprises mutations of a plurality of lysine and arginine residues between amino acids 697 and 716 of SEQ ID NO: 1. In some embodiments, the mutant comprises at least 1, 2, 3, 4, 5 or 6 mutations. Each possibility represents a separate embodiment of the invention. In some embodiments, in the mutant all lysines and arginines between amino acids 697 and 716 of SEQ ID NO: 1 are mutated. In some embodiments, the mutation is to a non-charged amino acid. In some embodiments, the mutation is to a non-polar amino acid. In some embodiments, the mutation is to a negatively charged amino acid. In some embodiments, the mutation is to alanine. In some embodiments, the cleavage resistant ACE2 comprises mutation of arginine 273 of SEQ ID NO: 1. In some embodiments, arginine 273 is mutated to alanine.

In some embodiments, the coronavirus is an alpha coronavirus. In some embodiments, the coronavirus is 229E or NL63. In some embodiments, the coronavirus is a beta coronavirus. In some embodiments the coronavirus is 0C43, HKU1 or MERS-CoV. In some embodiments, the coronavirus is SARS-CoV-1. In some embodiments, the coronavirus is SARS-CoV-2. In some embodiments, the coronavirus is selected from SARS-CoV-1 and SARS-CoV-2.

In some embodiments, the MSC and/or extracellular vesicle comprises at least one exogenous membrane associated peptide comprising a fragment of a virus interacting protein. In some embodiments, the MSC and/or extracellular vesicle comprises a plurality of exogenous membrane associated peptide comprising a fragment of a virus interacting protein. In some embodiments, the MSC and/or extracellular vesicle comprises at least 1, 2, 3, 4, 5 or 6 different exogenous membrane associated peptide comprising a fragment of a virus interacting protein. In some embodiments, the MSC and/or extracellular vesicle comprises at least two different exogenous membrane associated peptide comprising a fragment of a virus interacting protein.

In some embodiments, the exogenous membrane associated peptide comprises an extracellular fragment of a viral protein priming protein. In some embodiments, the viral protein is a virus spike protein. In some embodiments, the fragment of a viral protein priming protein is capable of priming a viral protein. In some embodiments, the receptor and the priming protein bind and prime the same virus. In some embodiments, the receptor and the priming protein both bind and prime a coronavirus. In some embodiments, the receptor and the priming protein both bind and prime SARS-CoV-1. In some embodiments, the receptor and the priming protein both bind and prime SARS-CoV-2. In some embodiments, the viral protein priming protein is transmembrane protease, serine 2 (TMPRSS2). In some embodiments, the viral protein priming protein is neuropilin-1 (NRRP1). In some embodiments, the priming protein is proteolytic. In some embodiments, the priming protein cleaves the receptor.

In some embodiments, the viral protein priming protein is an inactive mutant of the viral protein priming protein. In some embodiments, inactive is inactive to prime. In some embodiments, in active is proteolytically inactive. In embodiments, inactivating comprises the expression of an inactivating molecule. In some embodiments, the inactivating molecule is a peptide-conjugated morpholino.

In some embodiments, the exogenous membrane associated protein comprises a fragment of a viral protein. In some embodiments, the viral protein is a viral surface protein. In some embodiments, the viral protein is a viral entry protein. In some embodiments, the viral protein is a spike protein. In some embodiments, SARS-CoV-2 spike protein (S1) comprises the amino acid sequence:

(SEQ ID NO: 2)
QCVNLTTRTQLPPAYTNSFTRGVYYPDKVFRSSVLH
STQDLFLPFFSNVTWFHAIHVSGTNGTKRFDNPVLP
FNDGVYFASTEKSNIIRGWIFGTTLDSKTQSLLIVN
NATNVVIKVCEFQFCNDPFLGVYYHKNNKSWMESEF
RVYSSANNCTFEYVSQPFLMDLEGKQGNFKNLREFV
FKNIDGYFKIYSKHTPINLVRDLPQGFSALEPLVDL
PIGINITRFQTLLALHRSYLTPGDSSSGWTAGAAAY
YVGYLQPRTFLLKYNENGTITDAVDCALDPLSETKC
TLKSFTVEKGIYQTSNFRVQPTESIVRFPNITNLCP
FGEVFNATRFASVYAWNRKRISNCVADYSVLYNSAS
FSTFKCYGVSPTKLNDLCFTNVYADSFVIRGDEVRQ
IAPGQTGKIADYNYKLPDDFTGCVIAWNSNNLDSKV
GGNYNYLYRLFRKSNLKPFERDISTEIYQAGSTPCN
GVEGFNCYFPLQSYGFQPTNGVGYQPYRVVVLSFEL
LHAPATVCGPKKSTNLVKNKCVNFNFNGLTGTGVLT
ESNKKFLPFQQFGRDIADTTDAVRDPQTLEILDITP
CSFGGVSVITPGTNTSNQVAVLYQDVNCTEVPVAIH
ADQLTPTWRVYSTGSNVFQTRAGCLIGAEHVNNSYE
CDIPIGAGICASYQTQTNSPRRAR.

In some embodiments, SARS-CoV-2 spike protein (S1) consists of SEQ ID NO: 2.

According to some embodiments, the exogenous membrane associated protein comprises an extracellular fragment of a viral receptor binding domain (RBD). According to some embodiments, the exogenous membrane associated protein comprises a viral RBD. In some embodiments, the fragment is the receptor binding motif of the RBD. In some embodiments, an MSC or EV comprising an RBD acts as a competitive inhibitor of the binding of the virus to its receptor. In some embodiments, an MSC or EV comprising an RBD is useful as a vaccine (e.g., generate antibodies towards the virus). According to some embodiments, the exogenous membrane associated protein comprises the receptor binding domain (RBD) of SARS-CoV-1 and SARS-CoV-2 or any domain of the virus (e.g., in the S protein) that can inhibit the binding of the virus to ACE2 or an alternative receptor). According to some embodiments, the exogenous membrane associated protein comprises the receptor binding domain (RBD) of SARS-CoV-1 or SARS-CoV-2. According to some embodiments, RBD comprises amino acid residues 331-524 of SARS-CoV-2 S protein (e.g., SEQ ID NO:2) or amino acid residues 318-510 of SARS-CoV-1 S protein. In some embodiments, the receptor binding motif of the RBD comprises amino acids 437-509 of SEQ ID NO: 2. In some embodiments, the receptor binding motif of the RBD consists of amino acids 437-509 of SEQ ID NO: 2. In some embodiments, the RBD comprises amino acids 319-541 of SEQ ID NO: 2. In some embodiments, the RBD consists of amino acids 319-541 of SEQ ID NO: 2. In some embodiments, the RBD comprises

(SEQ ID NO: 4)
RVQPTESTVRFPNITNLCPFGEVFNATRFASVYAWN
RKRISNCVADYSVLYNSASFSTFKCYGVSPTKLNDL
CFTNVYADSFVIRGDEVRQIAPGQTGKIADYNYKLP
DDFTGCVIAWNSNNLDSKVGGNYNYLYRLFRKSNLK
PFERDISTEIYQAGSTPCNGVEGFNCYFPLQSYGFQ
PTNGVGYQPYRVVVLSFELLHAPATVCGPKKSTNLV
KNKCVNF.

In some embodiments, the RBD consists of SEQ ID NO: 4.

In some embodiments, an MSC and/or extracellular vesicle of the invention comprises an exogenous nucleic acid molecule that inhibits virus replication, inhibits inflammation, inhibits fibrosis, inhibits expression of a viral protein priming protein or a combination thereof. In some embodiments, the exogenous nucleic acid molecule inhibits virus replication. In some embodiments, the exogenous nucleic acid molecule inhibits inflammation. In some embodiments, the exogenous nucleic acid molecule inhibits fibrosis. In some embodiments, the exogenous nucleic acid molecule inhibits expression of a viral protein. In some embodiments, the exogenous nucleic acid molecule inhibits expression of a viral protein priming protein.

In some embodiments, the exogenous nucleic acid molecule is selected from a microRNA (miR), an anti-miR, a small interfering RNA (siRNA), an antisense oligonucleotide (ASO) or an mRNA.

In some embodiments, the miR and/or an anti-miR are over-expressed in the MSC or EV derived therefrom.

In some embodiments, the miR is a miR mimic. In some embodiments, the miR mimic is not a molecule found in nature. In some embodiments, the mimic is chemically modified. In some embodiments, the chemical modification extends the half-life of the mimic. In some embodiments, chemically modified is a chemically modified backbone. Chemical modification is well known in the art and includes, for example, 2-MOE, LNA, PNA, phosphorothioate and the like.

In some embodiments, an anti-miR is an antagomir. In some embodiments, an anti-mir is a molecule not found in nature. In some embodiments, the anti-mir is chemically modified. In some embodiments, the ASO is a molecule not found in nature. In some embodiments, the ASO is chemically modified.

In some embodiments, the antisense oligonucleotide (ASO), siRNA or both targets viral non-coding RNA (ncRNA). In some embodiments, the ncRNA is from SARS-CoV-1. In some embodiments, the ncRNA is from SARS-CoV-2. In some embodiments, the ncRNA is nsp3.1. In some embodiments, the ncRNA is nsp3.2.

In some embodiments, the siRNA binds to and inhibits an mRNA encoding TNF-alpha converting enzyme (TACE). In some embodiments, the ASO binds to and inhibits an mRNA encoding TACE.

As used herein, an “exogenous miR”, refers to expression of a miR, miR mimic or other synthetic version of the miR that has been introduced into the cell. The cell may express an endogenous form of the miR, but this refers to an externally introduced synthetic form of the miR. In some embodiments, synthetic is comprising chemical modification.

In some embodiments, the miR inhibits expression of a viral protein priming protein. In some embodiments, the viral protein priming protein is TMPRSS2. In some embodiments, the miR that inhibits expression of a viral protein priming protein is selected from miR-98-5p, let-7, miR-4458, miR-4500 and miR-582, and any combination thereof. In some embodiments, the miR that inhibits expression of a viral protein priming protein is selected from miR-98-5p, let-7, and miR-4458. In some embodiments, the miR is selected from miR-98-5p, let-7, and miR-4458. In some embodiments, the miR is miR-98-5p. In some embodiments, the miR is let-7. In some embodiments, the miR is miR-4458.

In some embodiments, the miR or anti-miR inhibits and/or reduces fibrosis and inflammation. In some embodiments, the anti-miR is anti-miR-214. In some embodiments, the anti-miR is anti-miR-21.

In some embodiments, the miR inhibits and/or reduces inflammation. In some embodiments, the miR is selected from: miR-145, let-7, miR-1, miR-106, miR-103, miR-10, miR-107, miR-1185, miR-124, miR-1226, miR-1271, miR-128, miR-129, miR-1303, miR-17, mir-93, miR-20, miR-106, miR-155 and miR-130, and any combination thereof. In some embodiments, the miR or anti-miR is selected from the group consisting of anti-miR-21, anti-miR-214, miR-124, miR-145 or miR-27a, miR-21, miR-155, miR-20, miR-9, miR-506, miR-124, and miR-455. In some embodiments, the miR or anti-miR is selected from the group consisting of anti-miR-214, miR-124, miR-145 or miR-27a, miR-21, miR-155, miR-20, miR-9, miR-506, miR-124, and miR-455. In some embodiments, the miR or anti-miR is selected from the group consisting of anti-miR-21, miR-124, miR-145 or miR-27a, miR-21, miR-155, miR-20, miR-9, miR-506, miR-124, and miR-455. In some embodiments, the miR or anti-miR is selected from the group consisting of miR-29c, miR-328, miR-190, miR-532 and anti-miR-214.

In some embodiments, the miR or anti-miR is selected from the group consisting miR-124, miR-145, miR-20, miR-9, miR-506, miR-455, miR-27a, miR-29c, miR-328, miR-190, miR-532, and anti-miR-214. In some embodiments, the miR or anti-miR is selected from the group consisting miR-124, miR-145, miR-29c, miR-328, miR-190, miR-532, and anti-miR-214. In some embodiments, the miR or anti-miR is miR-124 and anti-miR-214. In some embodiments, the MSCs comprises miR-124 and anti-miR-214. In some embodiments, the EVs comprises miR-124 and anti-miR-214. In some embodiments, the miR is miR-29c. In some embodiments, the miR is miR-328. In some embodiments, the miR is miR-190. In some embodiments, the miR is miR-532. In some embodiments, the anti-miR is anti-miR-214. In some embodiments, the miR is miR-124. In some embodiments, the miR is miR-145.

In some embodiments, the miR inhibits and/or reduces inflammation, such as in a pathological condition associated with a hyper pro-inflammatory response (e.g., ARDS, cytokine storm and the like). In some embodiments, the miR is selected from the group consisting of miR-5′70-3p, miR-125a-5p, hsa-miR-124, hsa-miR-34, hsa-miR-132, hsa-miR-146a, hsa-miR-223, and hsa-let-7c.

In some embodiments, the miR or anti-miR inhibits and/or reduces viral replication. In some embodiments, the miR or anti-miR that inhibits viral replication is selected from miR-5197-3p, miR-511-3p, anti-miR-9 and miR-3914, and any combination thereof. In some embodiments, the miR or anti-miR that inhibits viral replication is selected from miR-124 and miR-145. In some embodiments, the miR is miR-124. In some embodiments, the miR is miR-145.

In some embodiments, the one or more miRs inhibits and/or reduces fibrosis. In some embodiments, the miR is miR-145. In some embodiments, the miR is miR-145, miR-29c, anti-miR-214 and miR-328. In some embodiments, the miR is miR-145, miR-29c, anti-miR-21 and miR-328. In some embodiments, the miR is miR-145, miR-29c, anti-miR-214, anti-miR-21 and miR-328.

In some embodiments, the one or more miRs or anti-miRs inhibits and/or reduces lung fibrosis and inflammation. In some embodiments, the one or more miRs or anti-miRs inhibits and/or reduces lung fibrosis. In some embodiments, the miR or anti-miRs is at least one miR or anti-miR selected from the group consisting of anti-miR-21, anti-miR-181, anti-miR-214, anti-miR-1246, anti-miR-199, or overexpression (OE) of miR 29c, 27a, 31, 124, 127 or 26a. In some embodiments, the miR or anti-miRs is at least one miR or anti-miR selected from the group consisting of miR-29c, miR-328, and anti-miR-214. In some embodiments, the miR or anti-miRs is at least one miR or anti-miR selected from the group consisting of miR-29c, miR-328, miR-190 and anti-miR-214. In some embodiments, the miR is miR-29c. In some embodiments, the miR is miR-328. In some embodiments, the miR is miR-190. In some embodiments, the anti-miR is anti-miR-214.

In some embodiments, the one or more miRs or anti-miRs inhibits and/or reduces cystic fibrosis. In some embodiments, the miR or anti-miRs is at least one miR or anti-miR selected from the group consisting of miR-29, anti-miR-214 and/or Eluforsen. In some embodiments, the miR is at least one miR selected from the group consisting of miR-29, anti-miR-21 and/or Eluforsen.

As used herein, Eluforsen refers to a 33 nt, single-stranded, fully phosphorothioated and fully 2′-O-methyl-modified oligonucleotide partly complementary to the p.Phe508de1-CFTR RNA, and based on the CF4 molecule (5′-AUCAUAGGAAACACCAAAGAUGAUAUUUUCUUU-3′; SEQ ID NO: 3). In some embodiments, any Eluforsen includes any molecule having the same effect as Eluforsen.

In some embodiments, the one or more oligonucleotide molecule inhibits and/or reduces liver fibrosis. In some embodiments, the oligonucleotide molecule is selected from miR-30a, miR-9, miR-92-3p, a tribbles pseudokinase 3 (TRIB3) silencing molecule. Non-limiting examples of TRIB3 silencing molecule include microRNA (miR), an anti-miR, a small interfering RNA (siRNA), and an antisense oligonucleotide (ASO).

In some embodiments, the one or more miRs or anti-miRs inhibit and/or reduces liver fibrosis. In some embodiments, the miR or anti-miR is at least one miR or anti-miR selected from the group consisting of anti-miR-214, anti-miR-21, anti-miR-199, anti-miR-130, anti-miR-31, anti-miR-103, anti-miR-144, anti-miR-1825, miR-30d, miR-140p, miR-532 or miR-190.

In some embodiments, the one or more miRs or anti-miRs inhibit and/or reduce kidney fibrosis. In some embodiments, the miR or anti-miR is at least one miR or anti-miR selected from miR-532, miR-190 and anti-miR-214. In some embodiments, the miR is miR-532. In some embodiments, the miR is miR-190. In some embodiments, the anti-miR is anti-miR-214.

In some embodiments, the MSC of the invention or an extracellular vesicle of the invention are administered in combination with an anti-viral agent. Non-limiting examples of anti-viral agents include metformin, hydroxychloroquine, and melatonin.

In some embodiments, an MSC of the invention or an extracellular vesicle of the invention further comprises an anti-viral agent. In some embodiments, a composition of the invention further comprises an anti-viral agent. In some embodiments, the antiviral agent is selected from a vaccine, a TMPRSS2 inhibitor, an ACE2 blocking agent, soluble ACE2, and an anti-inflammatory compound. In some embodiments, the anti-inflammatory compound is a cannabinoid. In some embodiments, the antiviral agent is a vaccine. In some embodiments, the antiviral agent is a TMPRSS2 inhibitor. In some embodiments, the antiviral agent is an ACE blocking agent. In some embodiments, the antiviral agent is a soluble ACE2. In some embodiments, the antiviral agent is an anti-inflammatory compound.

In some embodiments, the virus interacting protein is fused to an Fc portion of an immunoglobulin. In some embodiments, the ACE2 is fused to an Fc portion of an immunoglobulin.

In some embodiments, the ACE2 blocking agent is an anti-ACE2 antibody. In some embodiments, the antibody is a blocking antibody. In some embodiments, the antibody is non-cytotoxic. In some embodiments, the antibody is an immunoglobulin-G2 (IgG2) or IgG4 antibody.

In some embodiments, the anti-viral agent is a cannabinoid. In some embodiments, the cannabinoid is selected from cannabidiol (CBD), tetrahydrocannabinol (THC) and a combination thereof. In some embodiments, the cannabinoid is CBD.

According to some embodiments, the CBD comprises at least one of: cannabidiol-C4 (CBD-C4), cannabidiol (CBD-05), cannabidiol momomethyl ether, cannabidiolic acid (CBD-A), cannabigerolic acid (CBN-A), cannabigerol (CBG), cannabinol (CBN), cannabinolic acid (CBN-A), cannabichromenic acid (CBC-A), cannabichromene (CBC), cannabidivarin, cannabidiorcol, cannabidivarinic acid, CBD-V, THC-V and any combination thereof. In some embodiments, the cannabinoid is selected from CBD, cannabidiol momomethyl ether, cannabidiolic acid (CBD-A), cannabigerolic acid (CBN-A), cannabigerol (CBG), cannabinol (CBN), cannabinolic acid (CBN-A), cannabichromenic acid (CBC-A), cannabichromene (CBC), cannabidivarin, cannabidiorcol, cannabidivarinic acid, CBD-V, THC-V and any combination thereof In some embodiments, the CBD is selected from cannabidiol-C4 (CBD-C4), cannabidiol (CBD-C5).

According to some embodiments, the THC comprises at least one of: (−)-Δ⁹-tetrahydrocannabinol (THC), 2-carboxy-THC (THCA), an ester of THCA, 4-carboxy-THC (THCA-B), an ester of THCA-B, 11-OH-THCA, THC-11-oic acid, Δ8-THC-11-oic acid, 1,1-dimethylheptyl-Δ8-THC-7-oic acid or any combination thereof.

The cannabinoid may be any isolate or purity of cannabinoid. In some embodiments, the cannabinoid is an essentially pure cannabinoid. In some embodiments, the cannabinoid is an isolated cannabinoid. In some embodiments, the cannabinoid is not psychoactive.

In some embodiments, the anti-viral agent is metformin. According to some embodiments, the anti-inflammatory compound is metformin. According to some embodiments, the anti-inflammatory compound is curcumin.

In some embodiments, the present invention comprises an isolated extracellular vesicle of an MSCs of the invention. In some embodiments, the extracellular vesicle is an isolated extracellular vesicle. In some embodiments, the extracellular vesicle is a purified extracellular vesicle.

The term “extracellular vesicle” as used herein refers to membrane bound vesicles that can be divided into three main groups: exosomes, microvesicles and apoptotic bodies. Exosomes are small membrane vesicles of endocytic origin with a size of 50-100 nm. They can contain microRNAs (miRNA), long non-coding RNAs (lncRNA), mRNAs, DNA fragments, and proteins, which are shuttled from donor to recipient cells. In addition to their natural role in cell-cell interactions, exosomes can be loaded with various drugs and exogenous nucleic acids or proteins and deliver this cargo to different cells.

As used herein, the terms “extracellular vesicles” and “exosomes” are the same and used interchangeably. In some embodiments, the extracellular vesicle is any natural or synthetic vesicle having similar functionality and/or activity as the extracellular vesicles of the invention. None-limiting examples of such vesicle include microvesicles, liposomes, micelles and the like.

In some embodiments, the extracellular vesicles are cell-derived vesicles. In some embodiments, the extracellular vesicle is from a plant cell. In some embodiments, the extracellular vesicle is from a bacterial cell. In some embodiments, the extracellular vesicle is from an animal cell. In some embodiments, the extracellular vesicles are cell-derived vesicles secreted from MSCs. In some embodiments, the extracellular vesicles are milk derived vesicles. In some embodiments, the animal is a mammal. In some embodiments, the extracellular vesicles from a mammal are from milk.

In some embodiments, the extracellular vesicle is from an animal cell. In some embodiments, the animal cell is an MSC. In some embodiments, the extracellular vesicle is from an animal MSC cell and the exogenous membrane associated protein is added directly to the extracellular vesicle. Methods of adding exogenous molecules to vesicles are well known in the art and include but are not limited to mixing, passive diffusion, active diffusion, and membrane anchoring. Any such method may be used in practicing the invention. In some embodiments, membrane association is by charge interaction. In some embodiments, membrane association is not by charge interaction.

### Pharmaceutical Composition

In some embodiments, the present invention comprises a pharmaceutical composition comprising any one of: an MSC of the present invention, an extracellular vesicle of the present invention and a combination thereof.

In some embodiments, the pharmaceutical composition comprises an MSC of the present invention. In some embodiments, the pharmaceutical composition comprises an extracellular vesicle of the present invention. In some embodiments, the pharmaceutical composition comprises a combination of a MSC of the present invention and an extracellular vesicle of the present invention.

In some embodiments, the pharmaceutical composition further comprises a pharmaceutically acceptable carrier, excipient or adjuvant. As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Method of Treatment

In some embodiments, the present invention comprises a method of treating, preventing or ameliorating a viral infection in a subject in need thereof, the method comprising administering to a subject at least one of:
a. a pharmaceutical composition described herein;
b. a pharmaceutical composition comprising an MSC;
c. a pharmaceutical composition comprising an extracellular vesicle from an MSC; and
d. a pharmaceutical composition comprising isolated and purified extracellular vesicles;
thereby treating, preventing or ameliorating said viral infection.

In some embodiments, there is provided at least one of:
a. a pharmaceutical composition described herein;
b. a pharmaceutical composition comprising an MSC;
c. a pharmaceutical composition comprising an extracellular vesicle from an MSC; and
d. a pharmaceutical composition comprising isolated and purified extracellular vesicles;
   for use in treating, preventing or ameliorating a viral infection in a subject in need thereof.

In some embodiments, the present invention comprises a method of treating, preventing or ameliorating an inflammation in a subject in need thereof, the method comprising administering to a subject at least one of:
a. a pharmaceutical composition described herein;
b. a pharmaceutical composition comprising an MSC;
c. a pharmaceutical composition comprising an extracellular vesicle from an MSC; and
d. a pharmaceutical composition comprising isolated and purified extracellular vesicles;
thereby treating, preventing or ameliorating said inflammation in the subject.

In some embodiments, there is provided at least one of:
a. a pharmaceutical composition described herein;
b. a pharmaceutical composition comprising an MSC;
c. a pharmaceutical composition comprising an extracellular vesicle from an MSC; and
d. a pharmaceutical composition comprising isolated and purified extracellular vesicles;
   for use in treating, preventing or ameliorating inflammation in a subject in need thereof.

In some embodiments, the inflammation is a result of an infection such as a virus including but not limited to SARS-CoV-1 or SARS-CoV-2. In some embodiments, the inflammation is SARS-CoV-2 induced inflammation.

In some embodiments, the present invention comprises a method of treating, preventing or ameliorating fibrosis in a subject in need thereof, the method comprising administering to a subject at least one of:
a. a pharmaceutical composition described herein;
b. a pharmaceutical composition comprising an MSC;
c. a pharmaceutical composition comprising an extracellular vesicle from an MSC; and
d. a pharmaceutical composition comprising isolated and purified extracellular vesicles;
thereby treating, preventing or ameliorating said fibrosis in the subject.

In some embodiments, there is provided at least one of:
a. a pharmaceutical composition described herein;
b. a pharmaceutical composition comprising an MSC;
c. a pharmaceutical composition comprising an extracellular vesicle from an MSC; and
d. a pharmaceutical composition comprising isolated and purified extracellular vesicles;
   for use in treating, preventing or ameliorating fibrosis in a subject in need thereof.

In some embodiments, the fibrosis (e.g., lung fibrosis) is a result of ARDS. In some embodiments, the fibrosis (e.g., lung, cardiac, kidney fibrosis) is a result of cytokine storm. In some embodiments, the fibrosis (e.g., lung, cardiac, kidney fibrosis) is a result of a severe hyperinflammatory responses (e.g., to a virus including but not limited to SARS-CoV-1 or SARS-CoV-2). In some embodiments, the fibrosis (e.g., lung, cardiac, kidney fibrosis) is a result of a chronic pathological condition. In some embodiments, the fibrosis is SARS-CoV-2 induced fibrosis.

In some embodiments, the method comprises administering the pharmaceutical composition described herein. In some embodiments, the method comprises administering a pharmaceutical composition comprising an MSC. In some embodiments, the method comprises administering a pharmaceutical composition comprising an extracellular vesicle from an MSC. In some embodiments, the method comprises administering a pharmaceutical composition comprising isolated and purified extracellular vesicles. In some embodiments, the method comprises administering a pharmaceutical composition comprising a combination of these compositions. Each possibility represents a separate embodiment of the invention.

In some embodiments, the MSC is a UC MSC. In some embodiments, the MSC is a CH MSC.

In some embodiments, the MSC is an unmodified MSC. In some embodiments, the extracellular vesicle is an unmodified extracellular vesicle.

In some embodiments, the pharmaceutical composition comprises isolated extracellular vesicles. In some embodiments, the pharmaceutical composition comprises purified extracellular vesicles. In some embodiments, purified comprises a purity of at least 70, 75, 80, 85, 90, 95, 97, 99 or 100%. Each possibility represents a separate embodiment of the invention. In some embodiments, the pharmaceutical composition is devoid of cells. In some embodiments, the pharmaceutical composition is depleted of cells. In some embodiments, the pharmaceutical composition is devoid of non-MSC cells. In some embodiments, the pharmaceutical composition is depleted of non-MSC cells.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

The term “subject” as used herein refers to an animal, more particularly to non-human mammals and human organism. Non-human animal subjects may also include prenatal forms of animals, such as, e.g., embryos or fetuses. Non—limiting examples of non-human animals include: horse, cow, camel, goat, sheep, dog, cat, non-human primate, mouse, rat, rabbit, hamster, guinea pig, pig. In one embodiment, the subject is a human. Human subjects may also include fetuses. In one embodiment, a subject in need thereof is a subject afflicted with a fractured bone, a bone injury, diminished bone mass and/or bone abnormality.

As used herein, the terms “subject” or “individual” or “animal” or “patient” or “mammal,” refers to any subject, particularly a mammalian subject, for whom therapy is desired, for example, a human.

In some embodiments, a subject in need thereof is infected with a viral infection. In some embodiments, a subject in need thereof is infected with a coronavirus. In some embodiments, a subject in need thereof is infected with a coronavirus caused by SARS-CoV-1. In some embodiments, a subject in need thereof is infected with a coronavirus caused by SARS-CoV-2. In some embodiments, the viral infection is an infection by a virus bound by the virus binding protein.

The term “administering” as used herein refers to any method which, in sound medical practice, delivers a composition containing an active agent to a subject in such a manner as to provide a therapeutic effect. One aspect of the present subject matter provides for oral administration of a therapeutically effective amount of a composition of the present subject matter to a patient in need thereof. Other suitable routes of administration can include parenteral, subcutaneous, intravenous, intramuscular, anal, oral, intrathecal intranasal, or intraperitoneal. In some embodiments, the administration is intravenous. In some embodiments, the administration is intranasal. In some embodiments, the administration is intrathecal. In some embodiments, the administration is intramuscular. In some embodiments, the administration is oral. In some embodiments, the administration is by inhalation. In some embodiments, the administering is systemic administration or administration to a site of infection. In some embodiments, administration is systemic administration. In some embodiments, administration is administration to a site of infection. In some embodiments, administration is to the lungs.

The dosage administered will be dependent upon the age, health, and weight of the recipient, kind of concurrent treatment, if any, frequency of treatment, and the nature of the effect desired.

In some embodiments, the MSCs are allogenic to the subject. In some embodiments, the MSCs are autologous to the subject.

In some embodiments, use of extracellular vesicles is superior to use of cells. It will be understood that as extracellular vesicles do not contain cellular machinery, they cannot facilitate viral replication. Thus, if the virus binds the vesicle this is no chance of the virus infecting an extracellular vesicle and reproducing as there would be with a cell. An extracellular vesicle can thus act as a decoy to bind virus without allowing spread of the infection.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### Methods

Generation of EVs expressing exogenous surface proteins: Using the CD63-GFP plasmid, the relevant protein/fragments were inserted in place of the 2^{nd} extracellular loop of CD63. As a control RFP was also inserted into this site. MSCs were transfected with these modified plasmids and then transferred to serum-free medium. EVs were isolated using either the ExoQuick kit or by ultracentrifugation. The following proteins/fragments were inserted: full length wild-type human ACE2, mutant human ACE2 R273A, and the receptor binding domain (amino acids 437-509) of the SARS-CoV-2 S1 protein.

### EXAMPLES

### Example 1: EVs Expressing ACE2 Receptor Block Pseudovirus Entry

In order to test the ability of EVs from MSCs to block virus entry a pseudotyped SARS-Cov-2 viral particle was engineered. Replication restricted recombinant pseudotyped lentiviral particles were generated containing the S1 protein from SARS-CoV-2. The viral particles also encoded a luciferase reporter protein. The viral particles were incubated with human lung epithelial cells expressing ACE2 and viral entry was monitored by measuring the luciferase activity of the cells. The relative increased luciferase activity of cells incubated with viral particles was a sign of viral entry.

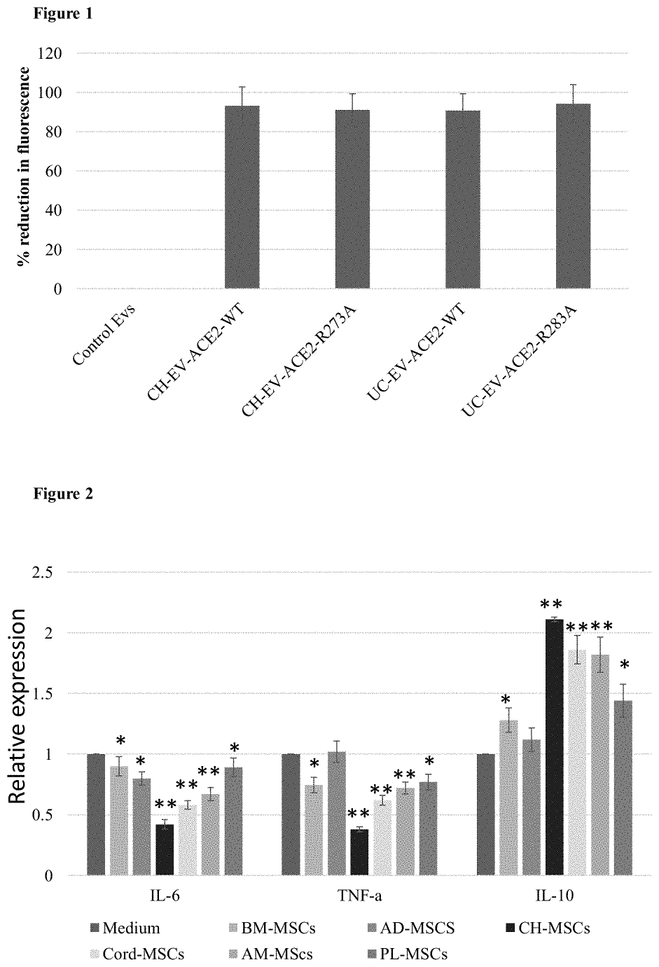

MSCs from chorionic placenta (CH) or from umbilical cord (UC) were transfected with an expression vector encoding either full length WT ACE2 protein or an ACE2 variant, R273A. Transduction with a lentiviral vector was also performed. EVs were isolated from the culture media of these cells and in order to test their effects on virus entry neutralization, the EVs were preincubated with the pseudovirus particles for 1 hour before they were incubated with the lung cells. EVs from non-transfected MSCs were used as a negative control. As can be seen in FIG. 1, all of the EVs containing ACE2 were highly effective at preventing virus entry, with a decrease in luciferase activity of at least 90%. In contrast, the unmodified EVs had no effect on virus entry.

EVs were also generated expressing the full SARS-CoV-2 receptor-binding-domain (RBD) of the SARS-CoV-2 RBD protein or the S1 peptide. These EVs were preincubated with the ACE2 expressing lung cells before the addition of the pseudovirus virus particles. These EVs protected the lung cells from infection in a comparable manner to the ACE2 expressing EVs.

### Example 2: MSCs and/or EVs for Treating ARDS, Cytokine Storm and Tissue Fibrosis

Acute respiratory distress syndrome (ARDS) is a lung injury caused by hyper pro-inflammatory response. One of the factors that contribute to ARDS, such as in subjects infected with SARS-CoV-2, is the hyperactivation of the immune system in response to virus infection. One of the important components of the immune system that contribute to the hyperactivation of the cytokine storm is macrophages.

As demonstrated hereinbelow, it was found that chorionic (CH) and umbilical cord (UC)-derived MSCs, and extracellular vesicles (EVs) secreted by these cells, induce high levels of the IL-10 in LPS-treated macrophages and decreased the secretion of IL-6 and TNF-alpha that contribute to cytokine storm. Similarly, these two MSC subpopulations induced higher expression of VEGF and KGF.

The ability of specific MSC and EV subpopulations to exert preferential effect on macrophage activation was tested. To mimic cytokine storm, LPS-treated macrophages were employed. THP-1 (human monocytes) were stimulated with PMA 100 nM to induce macrophage differentiation. The macrophages were then either co-cultured with different MSC subpopulations in transwell plates (1 mM filters) or with purified EVs isolated from MSC cultures, and this culture was followed by stimulation with LPS (20 ng/ml). Cytokine expression was measured by RT-PCR.

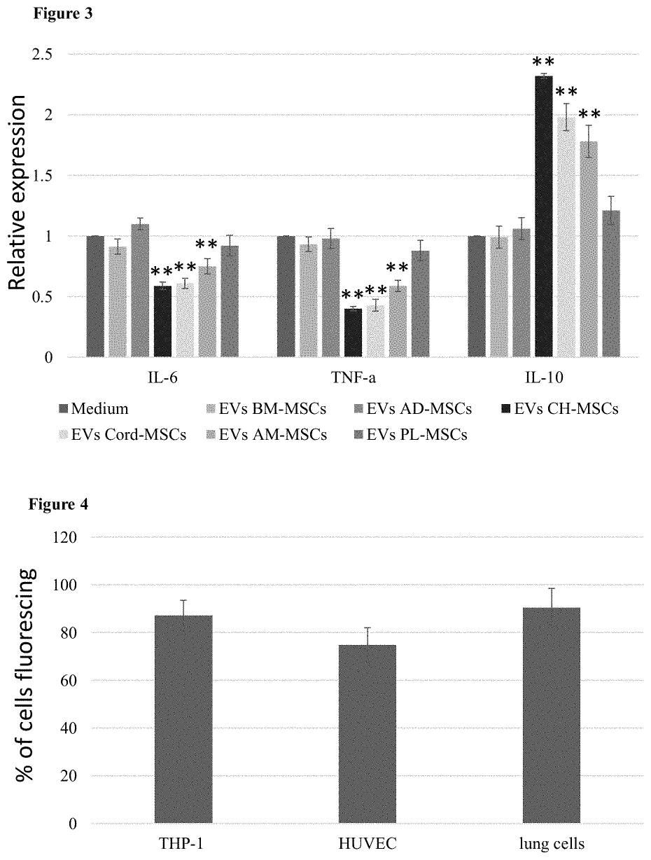

As presented in FIGS. 2 and 3, MSCs and EVs derived from chorionic placenta (CH) exerted the strongest anti-inflammatory and pro-M2 effects followed by umbilical cord (Cord) derived MSCs and EVs and then by amniotic membrane (AM) derived MSCs and EVs. Bone marrow (BM) and PL-MSCs (decidua basalis) derived cells also had an effect though it was greatly reduced, and adipose (AD) derived cells were the least effective. The extracellular vesicles (EVs) of these cells had comparable effects, with CH and Cord EVs having the greatest effect. These results demonstrate that choosing the right source of cells and EVs has important implications for the therapeutic effects of the cells on macrophages in general and cytokine storm in specific.

The function of the EVs is likely carried out by the delivery of cargo into the cytoplasm of target cells. By tracking fluorescent EVs from chorionic placenta MSCs it was determined that the vesicles are able to internalize their cargo into human macrophage, epithelial alveolar cells and HUVEC endothelial cells (FIG. 4).

Various combinations of miRs and anti-miRs expressed in EVs were tested for their ability to inhibit the proinflammatory response in macrophages. EVs expressing the combination of miR-124 and anti-miR-214 have the greatest potential in inhibiting pro-inflammatory response in LPS-activated THP-1 cells. These EVs inhibited the production of IL-6 by 81% and that of TNF-alpha by 77.65%. Similarly, EVs carrying miR-145 or miR-27a and anti-miR-214 exerted a similar effect. In contrast, the combination of miR-155 and anti-miR-214 did not reduce the expression of TNF-alpha and IL-6 but rather increased their expression by 19 and 22% respectively.

Further, it is shown that miRs inhibiting the NF-KB pathway include any one of miR-20, miR-9, miR-506, miR-124 or miR-455 in combination with miR-21 and miR-155 inhibit pro-inflammatory responses.

This Example shows the various inflammatory disorders associated with hyperactivation of the immune system, including but not limited to ARDS, sepsis, and cytokine storm, can be treated using subpopulations of MSCs and EVs.

Cord and placenta derived MSCs, specifically chorionic MSCs and/or their EVs may be administered locally, intravenously, intranasally, or by inhalation and decrease lung damage, inflammatory response, promote angiogenesis and regeneration.

### Example 3: MSCs and/or EVs for Treating COPD, Pneumonia and Emphysema

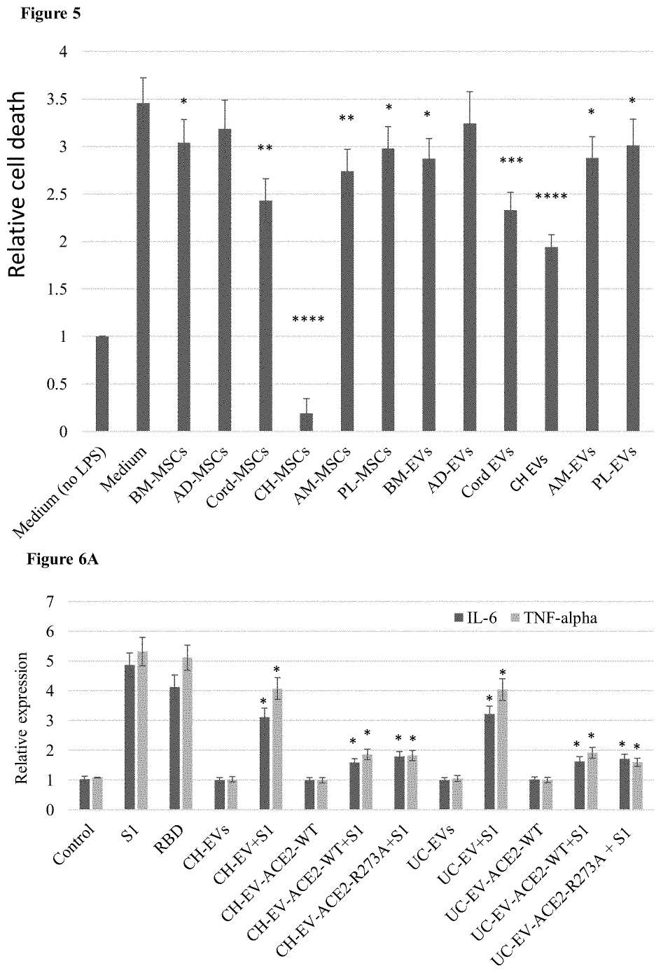

In order to test the ability of MSCs and their EVs to treat lung inflammation and chronic disease, lung endothelial cells were either co-cultured with different MSC subpopulations in transwell plates (1 mM filters) or with purified EVs isolated from MSC cultures. The cells were then stimulated with LPS (1 mg/ml) for 24 hr. Cell death was determined using the live/dead assay. As presented in FIG. 5, there was a differential protective effect of specific cells and EVs on the cells from LPS-induced cell death. CH-MSCs were so protective that they completely abrogated the effect of the LPS, and indeed cell death was reduced even as compared to the untreated control. Other than adipose (AD) MSCs, all other MSC types tested produced a protective effect, and their EVs were comparable. CH-MSC EVs were superior to all other cell types tested and all other EVs but were not as extraordinarily protective as the CH-MSCs themselves.

### Example 4: MSCs and/or EVs for Inhibiting SARS-CoV-2 Induced Inflammation

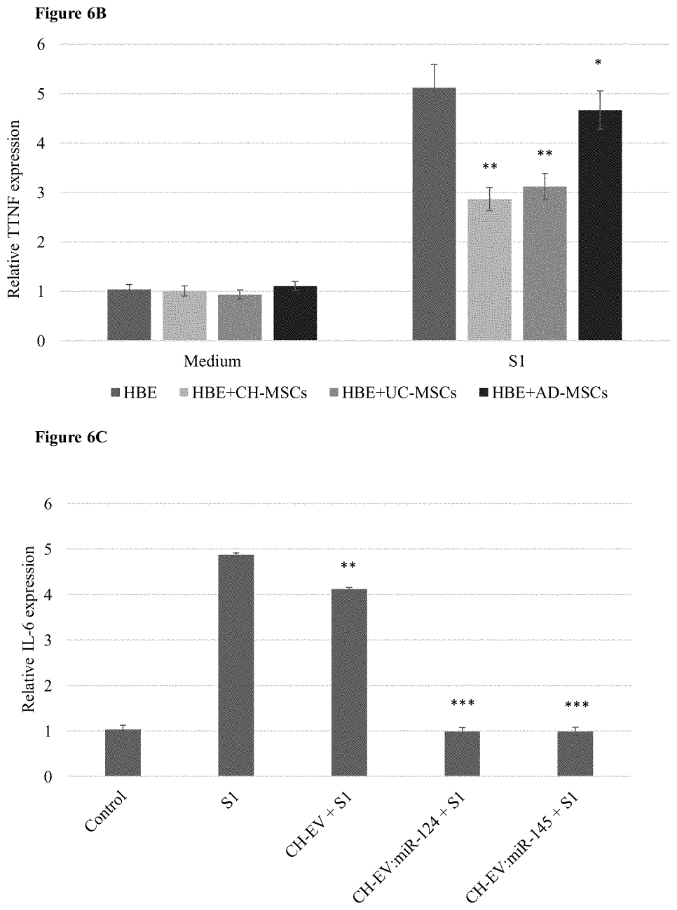

Immortalized human bronchial epithelial (HBE) cells were incubated with 100 ng/ml His-SARS-CoV-2 spike recombinant 51 subunit (51) or His-SARS-Cov-2-RBD (RBD) for 24 hr. Inflammation was monitored by measuring the expression of TNF alpha and IL-6. Both recombinant proteins produced an inflammatory effect with TNFα and IL-6 both increasing by 4-5 fold (FIG. 6A). EVs from unmodified CH-MSCs and UC-MSCs had a mild effect, reducing relative expression by ˜20-25%. EVs from MSC that were made to express either WT ACE2 or variant ACE2 were preincubated (1 hour) with the recombinant proteins before they were administered to the HBE cells. This produced a highly significant reduction in expression of the inflammatory markers with the levels returning nearly to normal (FIG. 6A). This indicates that the EVs were able to compete with the S1 protein's binding and activation of cellular ACE2. Similar results were observed regardless of whether WT or variant ACE2 was used.

Similarly, EVs expressing RBD were preincubated with the HBE cells before addition of the RBD recombinant protein. These EVs were also found to be highly effective at reducing inflammatory expression, as for example TNFα levels were reduced by 50% when EVs from CH-MSCs expression RBD were used.

These results were confirmed in a parallel system. The immortalized HBE cells were cocultured in transwell plates with various MSCs. To these cultures was added 100 ng/ml of recombinant S1 protein and the expression of TNFα was monitored (FIG. 6B). In the absence of MSCs, the S1 protein increased TNFα levels by 5-fold. CH- and UC-MSCs had a strong effect on this inflammatory marker, which AD-MSCs had a more modest effect.

Next it was tested if EVs loaded with anti-inflammatory miRs could also inhibit SARS-CoV-2 induced inflammation. HBE cells were incubated with the recombinant S1 as before and an increase in IL-6 expression was observed as before. When EVs from CH-MSCs expressing mimics of anti-inflammatory miRs (miR-124 or miR-145) were present in the culture the increase in IL-6 was completely abrogated (FIG. 6C). This indicates that EVs expressing inflammation inhibitor miRs can strongly inhibit the inflammation caused by SARS-CoV-2 infection.

### Example 5: MSCs and/or EVs for Inhibiting SARS-CoV-2 Induced Microglial Activation

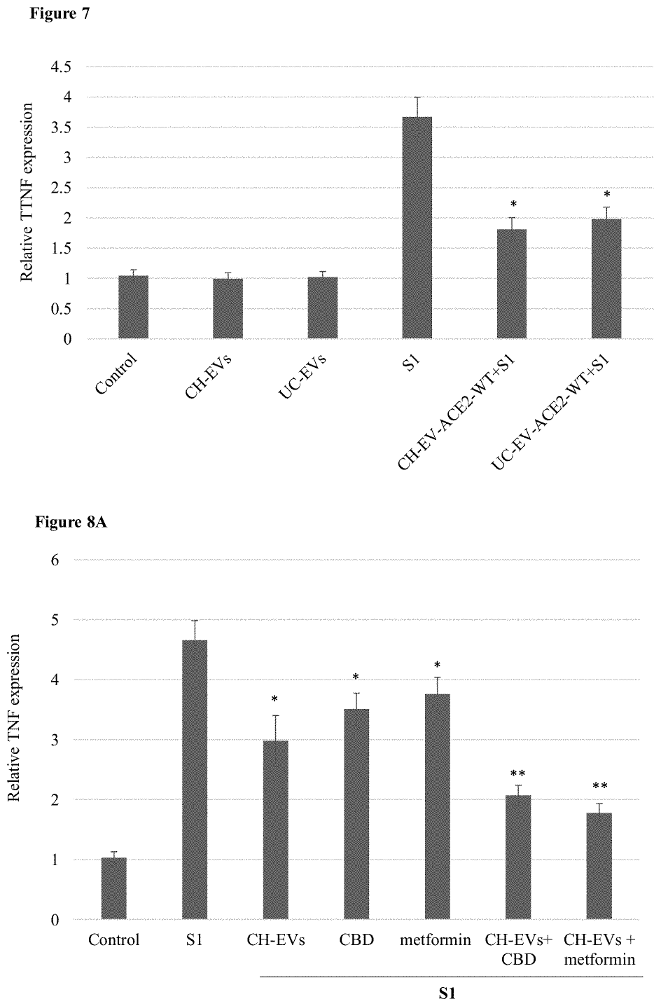

Immortalized human microglia cells (overexpressing ACE2) were incubated with 100 ng/ml His-SARS-CoV-2 spike recombinant S1 subunit (S1) for 24 hr. The expression of TNFα was determined by RT-PCR as a marker for microglial activation. The recombinant protein did activate the microglia producing a greater 3.5-fold increase (FIG. 7). EVs from MSC that were made to express WT ACE2 were preincubated (1 hour) with the recombinant proteins before they were administered to the microglial cells. This produced a highly significant reduction in expression of TNFα of ˜50% (FIG. 7). This indicates that the EVs were able to compete with the S1 protein's binding and activation of microglial ACE2.

### Example 6: Anti-Viral Combined Therapy using MSCs and/or EVs

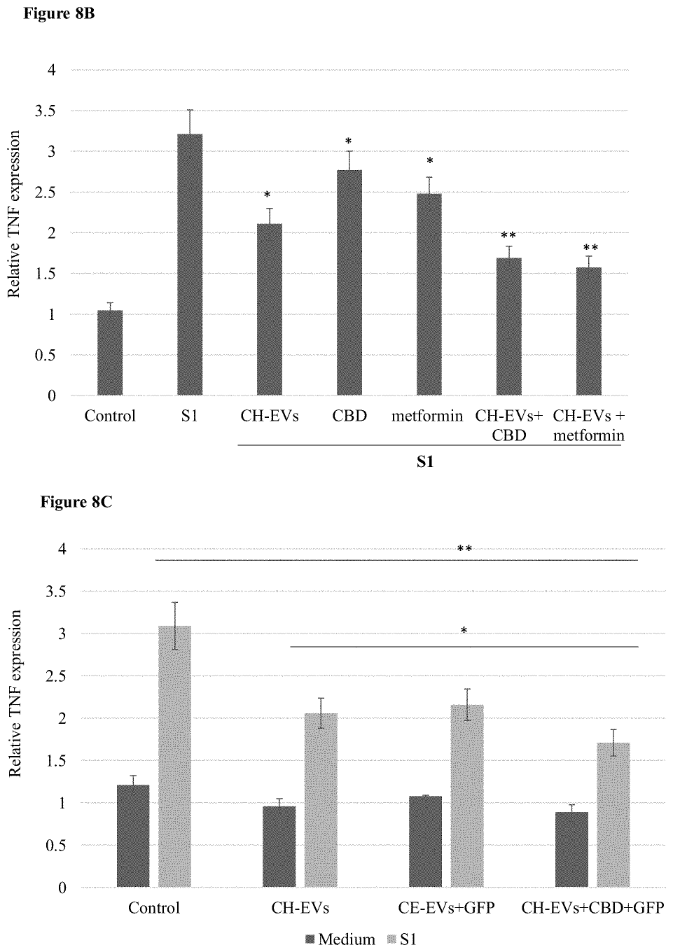

MSCs and EVs can be combined with different drugs (metformin, hydroxychloroquine, melatonin and other anti-viral drugs) to produce a more robust anti-viral effect. HBE cells were incubated with SARS-Cov-2 S1 peptide as before, and TNFα expression was quantified as a marker for inflammation. CH-EVs produced a modest reduction in inflammation as before, as did two different anti-viral treatments (CBD and metformin) when administered alone. The combination of the EVs with each treatment, however, produced a greater (although additive) response (FIG. 8A). Similar results were observed with immortalized human microglial cells overexpressing ACE2 (FIG. 8B).

Next, inclusion of the anti-viral drug within the EVs was tested. EVs from CH-MSCs were electroporated with either GFP alone or GFP and CBD. Fluorescence was monitored and it was determined that EVs were successfully loaded. Following electroporation, the EVs were added to immortalized human microglia alone or in the presence of S1 protein. TNF-alpha was determined 24 hr later by RT-PCR. As can be seen in FIG. 8C, unmodified EVs and EVs loaded with GFP behaved similarly and produced a modest, though significant reduction in TNFα. The EVs also expressing CBD produced an even stronger effect, which was significantly superior to the unmodified EVs. These data indicate that the EVs likely work through a distinct pathway from these two therapeutics and allow for a more robust therapeutic regimen for treating viral infections.

### Example 7: MSCs and/or EVs in the Treatment of Tissue Fibrosis

One of the complications of ARDS is lung fibrosis. Similarly, other organs such as heart and kidney have been also reported to be affected by cytokine storm and severe hyperinflammatory responses and in chronic pathological conditions. Therefore, tissue fibrosis represents a common pathological condition in various disorders.

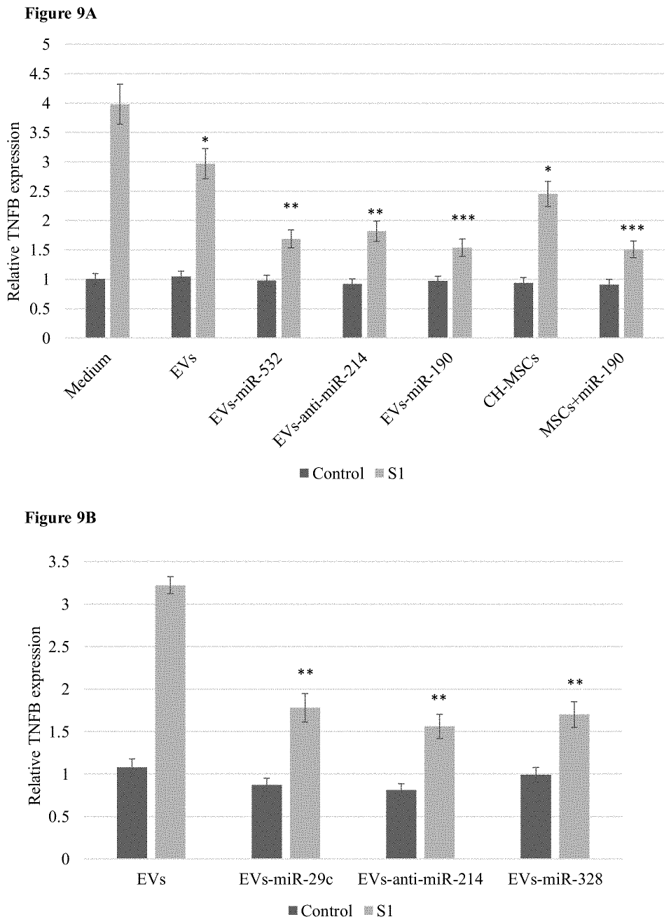

Kidney fibrosis: CH-EVs were loaded by electroporation with one of three anti-inflammatory molecules: miR-532, miR-190 or anti-miR-214. Human kidney fibroblasts were treated with recombinant S1 protein (100 ng/ml) for 24 hr with or without the loaded EVs. As presented in FIG. 9A, the S1 protein induced an increase in TGF-β (a marker of cell fibrosis) and unmodified CH-EVs produced a modest, though significant, reduction in this increase. EVs loaded with miR-532, miR-190 or an anti-miR-214 produced an even stronger inhibition on the fibrotic effect of S1. Further, when CH-MSCs were co-cultured with the fibroblasts, the unmodified MSCs also produced a reduction in TGF-β which was similarly enhanced with the CH-MSCs was loaded with miR-190 (FIG. 9A). These results demonstrate that unmodified EVs or CH-MSCs are effective at inhibiting fibrosis, but that over-expression of anti-inflammatory molecules can greatly enhance the effect.

Lung fibrosis: CH-EVs were loaded by electroporation with one of three anti-inflammatory molecules: miR-29c, miR-328 or anti-miR-214. Human lung fibroblasts were treated with recombinant S1 protein (100 ng/ml) for 24 hr with or without the electroporated CH-EVs. As presented in FIG. 9B, EVs loaded with miR-29c, miR-328 or an anti-miR-214 significantly inhibited the effect of S1. Similarly, CH-MSCs loaded with miR-190 also inhibited the expression of TGF-beta.

Lung fibrosis and inflammation—CH or cord MSCs or EVs carrying anti-miR-214, anti-miR-181, anti-miR-214, anti-miR-1246, anti-miR-199, or overexpressing miR-29c, miR-27a, miR-31, miR-124, miR-127 or miR-26a were highly effective.

Cystic fibrosis—MSCs or EVs with anti-fibrotic effects can be used for the treatment of cystic fibrosis. This MSCs or EVs can be loaded with the antisense oligonucleotide Eluforsen. Specifically, MSCs or MSC-derived exosomes carrying miR-29, and/or anti-miR-214 and/or Eluforsen are particularly effective.

Liver fibrosis—MSCs with agents that silence TRIM (mediates autophagy impairment) or their EVs are particularly effective. This silencing can be done using anti sense or siRNA, miR-30a, miR-9, and/or miR-92-3p.

Kidney fibrosis—MSCs or EVs derived from MSCs with an anti-inflammatory effect carrying antagomirs (anti-miRs) to the following miRs: miR-21, miR-214, miR-199, miR-130, miR-31, miR-103, miR-144, and miR-1825 are highly effective. MSCs or EVs carrying the following miRNAs: miR-30d, miR-140p, miR-532 or miR-190 are highly effective. Combinations of miRNA mimics or pre-miRNAs with anti-miRs is also highly effective.

This Example shows the tissue fibrosis, including but not limited to severe hyperinflammatory responses and in chronic pathological conditions following severe hyperinflammatory responses (e.g., ARDS, and cytokine storm) and in chronic pathological conditions, can be treated using subpopulations of MSCs and EVs.

### Example 8: MSCs and/or EVs for Inhibiting a Viral Protein Priming Protein

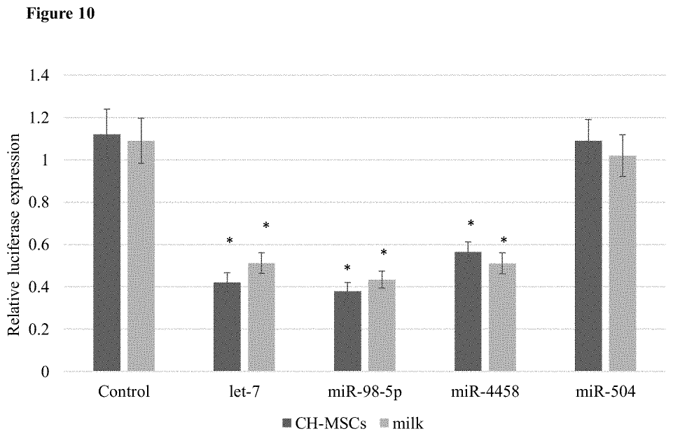

TMPRSS2 is known to function in combination with ACE2 to facilitate SARS-Cov-2 entry into cells. EVs isolated from CH-MSCs or bovine milk were loaded with various miRNA mimics by electroporation. These miRs were hypothesized to decrease TMPRSS2 expression and thus would enhance the anti-viral entry effect of the EVs. To test this, the EVs were incubated with HEK293 cells overexpressing a 3′-UTR of TMPRSS2 luciferase reporter plasmid. The EVs, regardless of whether they were from CH-MSCs or milk, that expressed let-7, miR-98-5p or miR-4458 were found to significantly inhibit luciferase expression, indicating that they do indeed target TMPRSS2 (FIG. 10). miR-504 expressing EVs (negative control) had no effect on luciferase expression.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A mesenchymal stem cell (MSC) or extracellular vesicle (EV) comprising an exogenous membrane associated peptide comprising a fragment of a viral peptide or virus-interacting protein.

2. The MSC or EV of claim 1, wherein said virus interacting protein is a virus binding receptor.

3. The MSC or EV of claim 1, wherein:
a. said MSC is an umbilical cord (UC) MSC or a chorionic placenta (CH) MSC;
b. said membrane associated peptide is a membrane embedded peptide comprising an extracellular domain of said virus interacting protein; or
c. both (a) and (b).

4. (canceled)

5. The MSC or EV of claim 2, wherein
a. said fragment of a virus binding receptor is capable of binding said virus;
b. said virus binding receptor is a receptor employed for viral entry; or
c. both.

6. (canceled)

7. The MSC or EV of claim 1, wherein said virus is a coronavirus and at least one of:
a. said virus binding receptor is angiotensin-converting enzyme 2 (ACE2);
b. said virus binding receptor is a cleavage resistant ACE2 mutant
c. said virus binding receptor is ACE2 comprising the amino acid sequence of SEQ ID NO: 1;
d. said virus binding receptor is an ACE mutant comprising SEQ ID NO: 1 comprising mutation of arginine 273 to alanine; and
e. said coronavirus is SARS-CoV-2.

8. (canceled)

9. (canceled)

10. (canceled)

11. The MSC or EV of claim 1, wherein:
a. said exogenous membrane associated protein comprises an extracellular fragment of a viral protein priming protein
b. said exogenous membrane associated protein comprises an extracellular fragment of a virus spike protein;
c. said exogenous membrane associated protein comprises an extracellular fragment of a viral protein priming protein is capable of priming said viral protein
d. said exogenous membrane associated protein comprises an extracellular fragment of a viral protein priming protein and said receptor and said priming protein bind and prime said virus;
e. said exogenous membrane associated protein comprises an extracellular fragment of transmembrane protease, serine 2 (TMPRSS2); or
f. said exogenous membrane associated protein comprises an extracellular fragment of an inactive mutant of a viral protein priming protein.

12. (canceled)

13. (canceled)

14. (canceled)

15. (canceled)

16. (canceled)

17. The MSC or EV of claim 1, wherein said viral peptide is a virus spike protein and said fragment comprises a receptor-binding domain (RBD) of said spike protein, optionally wherein said RBD comprises amino acids 437-509 of SEQ ID NO: 2.

18. (canceled)

19. The MSC or EV of claim 1, further comprising an exogenous microRNA (miR), anti-miR, small interfering RNA (siRNA), antisense oligonucleotide (ASO) or mRNA that inhibits virus replication, inhibits inflammation, inhibits fibrosis, inhibits expression of a viral protein priming protein or a combination thereof.

20. The MSC or EV of claim 19, wherein:
a. said viral protein priming protein is TMPRSS2 and said miR that inhibits expression of a viral protein priming protein is selected from miR-98-5p, let-7, and miR-4458;
b. said miR or anti-miR that inhibits inflammation is selected from: miR-124, miR-145, miR-20, miR-9, miR-506, miR-455, miR-27a, miR-29c, miR-328, miR-190, miR-532, and anti-miR-214;
c. said miR or anti-miR that inhibits inflammation is selected from: miR-124, miR-145, miR-29c, miR-328, miR-190, miR-532, and anti-miR-214; or
d. said MSC or EV comprises exogenous miR-124 and anti-miR-214.

21. (canceled)

22. (canceled)

23. (canceled)

24. The MSC or EV of claim 1, further comprising an anti-viral agent.

25. The MSC or EV of claim 24, wherein said antiviral agent is selected from a vaccine, a TMPRSS2 inhibitor, an ACE2 blocking agent, soluble ACE2, metformin, cannabidiol (CBD) and an anti-inflammatory compound.

26. (canceled)

27. The MSC or EV of claim 1, comprising at least two different membrane associated peptides comprising a fragment of a virus interacting protein.

28. The MSC or EV of claim 1, wherein at least one of:
a. said extracellular vesicle is from a plant cell, a bacterial cell, an animal cell or from milk;
b. said extracellular vesicle is from an MSC; and
c. said extracellular vesical is from an MSC of claim 1.

29. (canceled)

30. (canceled)

31. A mesenchymal stem cell (MSC) or an extracellular vesicle (EV) derived therefrom, comprising at least one subset of molecules selected from:
(i) at least one miR or anti-miR selected from the group consisting of miR-29c, anti-miR-214 and miR-328;
(ii) at least one miR or anti-miR selected from the group consisting of miR-29c, anti-miR-214, miR-328 and miR-190;
(iii) at least one miR or anti-miR selected from the group consisting of anti-miR-181, anti-miR-214, anti-miR-1246, anti-miR-199, miR-29c, miR-27a, miR-31, miR-124, miR-127 and miR-26a;
(iv) at least one molecule selected from the group consisting of miR-29, anti-miR-214 and Eluforsen;
(v) at least one molecule selected from miR-30a, miR-9, miR-92-3p, a TRIB3 silencing molecule;
(vi) at least one miR or anti-miR selected from the group consisting of anti-miR-214, anti-miR-214, anti-miR-199, anti-miR-130, anti-miR-31, anti-miR-103, anti-miR-144, anti-miR-1825, miR-30d, miR-140p, miR-532 or miR-190;
(vii) at least one miR or anti-miR selected from the group consisting of anti-miR-214, miR-124, miR-145 or miR-27a, miR-21, miR-155, miR-20, miR-9, miR-506, miR-124, miR-455;
(viii) at least one miR or anti-miR selected from miR-532, miR-190 and anti-miR 214; and
(ix) any combination thereof.

32. The MSC or EV of claim 31, comprising at least one of:
a. at least one miR or anti-miR selected from the group consisting of miR-29c, anti-miR-214, miR-190 and miR-328 and has an anti-fibrotic effect in the lung;
b. at least one miR or anti-miR selected from the group consisting of anti-miR-214, miR-190 and miR-532 and has an anti-fibrotic effect in the kidneys; and
c. at least one molecule selected from the group consisting of miR-29, anti-miR-214 and/or Eluforsen.

33. (canceled)

34. (canceled)

35. (canceled)

36. (canceled)

37. (canceled)

38. A pharmaceutical composition comprising:
an MSC of claim 1 and a pharmaceutically acceptable carrier or adjuvant.

39. (canceled)

40. A method of treating, preventing or ameliorating a viral infection in a subject in need thereof, the method comprising administering to said subject:
a pharmaceutical composition of claim 38;
thereby treating, preventing or ameliorating said viral infection.

41. The method of acclaim 40, wherein at least one of:
a. said MSC is selected from a UC MSC, and a CH MSC;
b. said MSC is allogenic or autologous to said subject and
c. said viral infection is an infection by a virus bound by said virus binding protein.

42. (canceled)

43. The method of any one of claim 40, wherein said administering is
a. systemic administration or administration to a site of infection;
b. selected from inhalation, intravenous, intranasal, intrathecal, intramuscular or oral administration; or
c. both (a) and (b).

44. (canceled)

45. (canceled)

46. (canceled)

47. The method of claim 40, further comprising administering an anti-viral medication, optionally wherein said anti-viral medication is selected from CBD and metformin.

48. (canceled)
